(19) United States
(12) Patent Application Publication
(10) Pub. No.: US 2011/0136791 A1
(43) Pub. Date: Jun. 9, 2011

(54) COMBINATION OF A NICOTINIC RECEPTOR PARTIAL AGONIST AND OF AN ACETYLCHOLINESTERASE INHIBITOR, PHARMACEUTICAL COMPOSITION CONTAINING SAME AND USE THEREOF IN THE TREATMENT OF COGNITIVE DISORDERS

(75) Inventors: Olivier BERGIS, Gif-sur-Yvette (FR); Philippe PICHAT, Orsay (FR); Alexandre URANI, Bourg-la-reine (FR)

(73) Assignee: SANOFI-AVENTIS, Paris (FR)

(21) Appl. No.: 12/958,025

(22) Filed: Dec. 1, 2010

Related U.S. Application Data
(63) Continuation of application No. PCT/FR2009/051041, filed on Jun. 2, 2009.

(30) Foreign Application Priority Data
Jun. 2, 2008 (FR) 0802996

(51) Int. Cl. A61K 31/551; A61K 31/445; A61K 31/27; A61P 25/00; A61P 25/28

(52) U.S. Cl. 514/221; 514/319; 514/479

(57) ABSTRACT

The combination of an alpha-7 nicotinic receptor agonist and of an acetylcholinesterase inhibitor, pharmaceutical composition comprising the combination and the use thereof in the treatment of cognitive disorders.

This application is a continuation of International Application No. PCT/FR2009/051041, filed Jun. 2, 2009, which is incorporated herein by reference in its entirety; which claims the benefit of priority of French Patent Application No. 0802996 filed Jun. 2, 2008.

The invention relates to the combination of an alpha-7 nicotinic receptor partial agonist and of an acetylcholinesterase inhibitor, to a pharmaceutical composition comprising the combination of the invention and to the use thereof in the treatment of cognitive disorders, and more particularly Alzheimer's-disease-related cognitive disorders.

The term “cognitive disorders” is intended to mean deficits in higher intellectual functions which concern, inter alia, short-term and long-term memory disorders, working memory disorders, attention and vigilance process disorders, semantic memory disorders, spatial memory disorders, and higher executive function (abstraction and planning, judgment) disorders.

Patients suffering from Alzheimer's-disease-related cognitive disorders are currently treated by administering acetylcholinesterase inhibitors.

One of the difficulties in treating Alzheimer's-disease-related cognitive disorders with acetylcholinesterase inhibitors lies in the fact that many treated patients gradually develop adverse side effects as the administrations are repeated. Thus, 10 to 15% of treated patients are obliged to interrupt the treatments owing to the gastrointestinal side effects (nausea, vomiting, diarrhea) and 40% of treated patients cannot receive an optimum therapeutic dose because of these side effects. In addition, only 30 to 50% of treated patients will respond to the treatment, and the therapeutic effects observed often consist only of a simple moderate stabilization of the symptoms (for a review, see the report Alzheimer's disease, Decision Resources, USA, June 2006). It is consequently necessary to find medicaments which are more effective *Alzheimer's-disease-related cognitive disorders, and which in addition exhibit fewer adverse side effects.

The invention aims to reply to this technical problem by proposing a combination of an alpha-7 nicotinic receptor partial agonist and of an acetylcholinesterase inhibitor.

Alpha-7 nicotinic receptor (a7-nAChR) partial agonists have been described as having pro-cognitive properties in several animal models which examine various types of memory or cognitive functions (Biton et al., Neuropsychopharmacology, 2007, 32: 1-16; Pichat et al., Neuropsychopharmacology, 2007, 32: 17-34).

The use of a partial agonist and not of a full agonist represents a significant technological advance. This is because the alpha-7 nicotinic receptor is known to exhibit rapid and pronounced desensitization phenomena. A partial agonist, unlike a full agonist, will only very slightly desensitize the receptor, thereby resulting in persistence of the therapeutic effects as the treatments are repeated.

The combination of the invention exhibits an improved action compared with the action of the two active ingredients taken individually. Specifically, a greater effect of the combination on the size of the pro-cognitive pharmacological effect is observed compared with the effect produced of the two active ingredients taken individually. This greater effect of the combination would allow:
the prescribed dose to be reduced and therefore allows better tolerance to the treatment (reduction in gastrointestinal side effects);
the possibility of a longer patient treatment period, owing to the better tolerance;
an increase in the percentage of patients responding to the combination treatment compared with the treatment with the acetylcholinesterase inhibitors alone.

A first subject of the invention relates to a combination of an alpha-7 nicotinic receptor partial agonist and of an acetylcholinesterase inhibitor.

A second subject of the invention relates to a pharmaceutical composition comprising, as active ingredient, such a combination.

A third subject of the invention relates to the use of such combination for the treatment of cognitive disorders of varied origins, and more particularly Alzheimer's-disease-related cognitive disorders.

Thus, a first subject of the invention relates to the combination of an alpha-7 nicotinic receptor partial agonist and of an acetylcholinesterase inhibitor.

Among the alpha-7 nicotinic receptor partial agonists of the invention, mention may be made of the alpha-7 nicotinic receptor partial agonist of general formula (I)

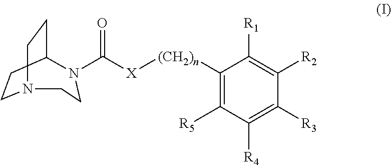

in which:
X represents an oxygen atom or a group of formula NZ in which Z represents a hydrogen atom or a (C₁-C₆)alkyl group,
n represents the number 0, 1 or 2, and
R₁, R₂, R₃, R₄ and R₅ each represent, independently of one another, a hydrogen or halogen atom, or a trifluoromethyl group, a trifluoromethoxy group, a cyano group, a hydroxyl group, a (C₁-C₆)alkyl group, a (C₁-C₆)alkoxy group, a phenoxy group or a phenyl group which is optionally substituted with a halogen atom or a trifluoromethyl group, a cyano group, a hydroxyl group, a (C₁-C₆)alkyl group or a (C₁-C₆)alkoxy group, or else R₂ and R₃ together form a group of formula —OCH₂O— or —CH₂CH₂CH₂CH₂—,
in the form of a base or of an addition salt with an acid, of a hydrate or of a solvate.

The compounds of general formula (I) are described in document WO 00/58311.

### BRIEF DESCRIPTION OF THE DRAWINGS

FIG. 1 shows the time spent in locomotion during the first accustomization session for each of the experimental groups in a test for spontaneous forgetting in rats with no deficit.

FIG. 2 shows the exploration time (in seconds) for the two objects, namely the new one and the familiar one in a test for spontaneous forgetting in rats with no deficit.

FIG. 3 shows the recognition index during the recalling session for each of the experimental groups for a test for spontaneous forgetting in rats with no deficit.

FIG. 4 shows the exploration time (in seconds) for the two objects, namely the new object and the familiar object for a test of short-term visual episodic memory deficit.

FIG. 5 shows the recognition index during the recalling session for each of the experimental groups for a test of short-term visual episodic memory deficit.

In the context of the present invention,
the expression “Cₜ-C_{Z} where t and z can have the values of 1 to 6” is intended to mean: a carbon chain which can have from t to z carbon atoms; for example, the term “C₁-C₃” is intended to mean a carbon chain which can have from 1 to 3 carbon atoms;
the term “a halogen atom” is intended to mean: a fluorine, chlorine, bromine or iodine atom;
the term “an alkyl group” is intended to mean: a linear or branched, saturated aliphatic group. By way of examples, mention may be made of methyl, ethyl, propyl, isopropyl, butyl, isobutyl, tert-butyl, pentyl, etc., groups;
the term “an alkoxy group” is intended to mean: an —O-alkyl radical of which the alkyl group is as defined above.

The compounds of general formula (I) defined above have been described in document WO 00/58311 as alpha-7 nicotinic receptor partial agonists.

The salts of the compounds of general formula (I) that can be used according to the invention can be prepared with pharmaceutically acceptable acids, but the salts of other acids that are useful, for example, for purifying or isolating the compounds of formula (I) are also part of the invention.

The compounds of general formula (I) can exist in the form of hydrates or of solvates, i.e. in the form of combinations or associations with one or more molecules of water or with a solvent. Such hydrates and solvates are also part of the invention.

The compounds of general formula (I) can comprise one or more asymmetric carbon atoms. They can therefore exist in the form of enantiomers or diastereoisomers. These enantiomers and diastereoisomers, and also mixtures thereof, including racemic mixtures, are part of the invention.

A first group of compounds of formula (I) that can be used according to the invention is that in which X represents an oxygen atom.

A second group of compounds of formula (I) that can be used according to the invention is that in which n represents the number 0.

A third group of compounds of formula (I) that can be used according to the invention is that in which R₁, R₂, R₃, R₄ and R₅ each represent, independently of one another, a hydrogen or halogen atom.

A fourth group of compounds of the formula (I) that can be used according to the invention is that in which R₁, R₂, R₄ and R₅ each represent a hydrogen atom and R₃ represents a halogen atom.

A fifth group of compounds of the formula (I) that can be used according to the invention is that in which

X represents an oxygen atom;
n represents the number 0;
R₁, R₂, R₄ and R₅ each represent a hydrogen atom and
R₃ represents a halogen atom.

Among the compounds of formula (I) that can be used in the context of the invention, mention may in particular be made of 4-bromophenyl 1,4-diazabicyclo[3.2.2]nonane-4-carboxylate, in the form of a base of an addition salt with an acid, of a hydrate or of a solvate.

An example of a salt of 4-bromophenyl 1,4-diazabicyclo[3.2.2]nonane-4-carboxylate that can be used according to the invention is the fumarate ((2E)-but-2-enedioate) salt.

In accordance with the invention, the compounds of general formula (I) can be prepared according to the process described in application WO 00/58311.

### EXAMPLE

### Preparation of 4-bromophenyl 1,4-diazabicyclo[3.2.2]nonane-4-carboxylate (2E)-but-2-enedioate

232.45 g of 4-bromophenyl 1,4-diazabicyclo[3.2.2]nonane-4-carboxylate (WO 00/58311) are placed in 2331 ml of ethanol, in a 6 l jacketed reactor. The reaction medium is brought to 60° C. A solution of 87.17 g of fumaric acid in 1000 ml of ethanol and 97 ml of water, preheated to 60° C., is then added. 1900 ml of solvent are subsequently distilled off and then the reaction medium is cooled to 20° C. over 2.5 h. After 2.5 h of contact at 20° C., the 4-bromophenyl 1,4-diazabicyclo[3.2.2]nonane-4-carboxylate (2E)-but-2-enedioate is filtered, washed with twice 400 ml of ethanol, and then dried under vacuum at 50° C.

Melting point (DSC): 175° C.

¹H NMR (400 MHz, DMSO-d6) δ (ppm): 1.80 (bs^{(a)}, 2H), 2.10 (bs, 2H), 3.10 (bs, 6H), 3.71 (bs, 1H), 3.84 (bs, 1H), 4.25 (bs, 0.5H), 4.40 (bs, 0.5H), 6.58 (s, 2H), 7.14 (m, 2H), 7.57 (m, 2H), 11 (bs, 2H).(a) bs=broad singlet.

For the purposes of the present invention, the acetylcholinesterase inhibitor can be chosen from all the acetylcholinesterase inhibitors known in the literature. By way of example, mention may in particular be made of rivastigmine (Exelon®) and donepezil (Aricept®).

Thus, an example of a combination according to the invention is the combination consisting of 4-bromophenyl 1,4-diazabicyclo[3.2.2]nonane-4-carboxylate (2E)-but-2-enedioate and of rivastigmine.

Another example of the combination according to the invention is the combination consisting of 4-bromophenyl 1,4-diazabicyclo[3.2.2]nonane-4-carboxylate (2E)-but-2-enedioate and of donepezil.

A second subject of the invention relates to a pharmaceutical composition comprising, as active ingredient, a combination as defined above, and one or more pharmaceutically acceptable excipients.

The pharmaceutical composition contains a minimum active dose of each active ingredient present in the combination according to the invention.

The invention also relates to a pharmaceutical composition containing a subactive dose of each active ingredient present in the combination according to the invention. The use of such subactive doses can make it possible to avoid the side effects of one or more active ingredients present in the combination according to the invention.

The excipients are chosen, according to the pharmaceutical form and the mode of administration desired, from the usual excipients which are known to those skilled in the art.

The composition can be administered orally, parenterally or rectally.

Suitable unit administration forms comprise oral administration forms such as tablets, soft or hard gel capsules, powders, granules and oral solutions or suspensions, sublingual, buccal, intracheal, intraocular and intranasal administration forms, forms for administration by inhalation, topical, transdermal, subcutaneous, intramuscular or intravenous administration forms, rectal administration forms, and implants. For topical application, the active ingredients according to the invention can be used in creams, gels, ointments or lotions.

According to the invention, the two active ingredients are administered according to the same route, for example orally, or of the active ingredients is administered according to a first route, for example orally, and the other active ingredient is administered according to a different route, for example parenterally.

When a composition in tablet form is prepared, the active ingredients are mixed with one or more pharmaceutical excipients, such as gelatin, starch, lactose, magnesium stearate, talc, silica, gum arabic, mannitol, microcrystalline cellulose, hypromellose, or the like.

The tablets can be coated with sucrose, with a cellulosic derivative or with other materials suitable for coating. The tablets can be prepared by various techniques, such as direct compression, dry or wet granulation or hot melt.

It is also possible to obtain a pharmaceutical composition in gel capsule form by mixing the active ingredients with a diluent and pouring the mixture obtained into soft or hard gel capsules.

For parenteral administration, use is made of aqueous suspensions, isotonic saline solutions or sterile injectable solutions which contain pharmacologically compatible agents, for example propylene glycol or butylene glycol.

According to the usual practice, the dosage suitable for each patient is determined by the physician according to the mode of administration, and the age, weight and response of said patient.

The doses depend on the desired effect, on the duration of treatment and on the route of administration used.

For example, with oral administration, the daily doses of each of the active ingredients of the combination according to the invention are the following:
alpha-7 nicotinic receptor partial agonists: between 0.5 and 500 mg per day and per person, in particular between 1 and 100 mg per day and per person;
acetylcholinesterase inhibitor: between 3 and 200 mg per day and per person, and in particular between 5 and 100 mg per day and per person.

There may be particular cases where higher or lower dosages are suitable. Such dosages do not depart from the context of the invention.

The respective doses of the alpha-7 nicotinic receptor partial agonist and of the acetylcholinesterase inhibitor are generally approximately identical to one another or else can differ from one another.

By way of example, a unit administration form of the alpha-7 nicotinic receptor partial agonist in tablet form comprises the following ingredients:

| | |
|---|---|
| Compound | 4 mg |
| Mannitol | 174 mg |
| Sodium croscarmellose | 6 mg |
| Corn starch | 15 mg |
| Hydroxypropylmethylcellulose | 2 mg |
| Magnesium stearate | 3 mg |

Also by way of example, a unit administration form of the acetylcholinesterase inhibitor in tablet form can comprise 10 mg of the acetylcholinesterase inhibitor and possible excipients, for example microcrystalline cellulose, colloidal silica, talc, sodium croscarmellose and magnesium stearate.

The administration of each of the active ingredients can also be carried out simultaneously, separately or spread out over time (sequential administration).

When the administration is carried out simultaneously, the two active ingredients can be combined within a single pharmaceutical composition, comprising the two active ingredients, such as a table or a gel capsule.

The two active ingredients can also, irrespective of whether or not their administration is simultaneous, be present in separate pharmaceutical compositions. To this effect, the combination according to the invention may be in the form of a kit comprising, firstly, at least one alpha-7 nicotinic receptor partial agonist as defined above and, secondly, at least one acetylcholinesterase inhibitor as defined above, the alpha-7 nicotinic receptor partial agonist and the acetylcholinesterase inhibitor being in separate compartments and being intended to be administered simultaneously, separately or spread out over time (sequential administration).

Another subject of the invention relates to the use of a combination as described above, for the preparation of a medicament intended for the treatment of cognitive disorders, and more particularly Alzheimer's-disease-related cognitive disorders.

A subject of the invention is also a method for treating cognitive disorders, and more particularly Alzheimer's-disease-related cognitive disorders, which comprises the administration, to a patient, of an active or subactive minimum dose of an alpha-7 nicotinic receptor partial agonist as defined above, and of an active or subactive minimum dose of an acetylcholinesterase inhibitor as defined above, said doses being administered simultaneously, separately, or sequentially, as described above.

The effects of the combination on the cognitive disorders have been evaluated by means of two behavioral psychopharmacology tests in rodents (rats and mice). These tests are aimed at one of the main types of cognitive disorder, namely memory disorders, and more particularly visual episodic memory disorders. The objective is to demonstrate the greater effect of the combination on the size of the pro-cognitive pharmacological effect compared with the effect produced by the two active ingredients taken individually, in the treatment of these cognitive disorders.

1. Test for Spontaneous Forgetting in Rats with No Deficit

A first study aimed to demonstrate, in adult rats, a procognitive synergy of the combination according to the invention in a visual episodic memory task using a spontaneous forgetting protocol in the object recognition test.

1.) Accustomization phase: In this protocol, the animals are first subjected to a session of accustomization to the environmental context (a wooden chamber of 65×45×45 cm) for a period of 2 minutes. The time spent in active locomotion is measured using a timer.
2.) Learning phase: 24 hours later, two exactly identical objects (either metal triangles or pyramids made of Lego) are placed in the experimental device. Each rat is placed in the middle of the chamber, the nose against the wall opposite that where the objects/stimulus are located. The pairs of objects presented are alternated equitably in each of the groups of rats. The animals are left in the experimental chamber until they reach a period of exploration of the two objects equal to 20 seconds, out of a maximum period of 3 minutes. As soon as the 20 seconds of exploration are reached, the rat is put back in its housing cage. This procedure makes it possible to homogenize the degree of impregnation of the animals for the familiar objects, and therefore to obtain a degree of learning that is comparable from one animal to the other.
3.) Recalling session: The recalling session takes place after a forgetting period of 24 h for all the groups. The rats are confronted with a pair of objects consisting of the object encountered during the learning phase and an unknown object. The rats are placed in the same way as for the learning session.

This test lasts 3 minutes, during which the time spent exploring each of the 2 objects is measured.

If the animal shows significant recollection of the previously memorized object, it will explore predominantly the new object. Conversely, if the animal has forgotten the familiar object, the times spent exploring the new object and the familiar object will be similar.

For the learning and recalling sessions, the exploring of an object is counted if the animal orients its nose toward the object, inside a perimeter of 2 centimeters around said object, or if it touches it with its nose or with its feet. If the rat moves around the object or sits on it, these behaviors are not considered to be exploratory.

The raw data are expressed in seconds.

The test compounds are administered orally 60 minutes (rat study) before each of the three sessions of the protocol.

Four experimental groups are formed, said groups receiving, respectively:

1—vehicle (methylcellulose+Tween alone)
2—alpha-7 nicotinic receptor partial agonist (inactive dose)
3—acetylcholinesterase inhibitor (inactive does)
4-alpha-7 nicotinic receptor partial agonist (inactive dose)+acetylcholinesterase inhibitor (inactive dose).

During the recalling session, 24 hours after the learning session, the times spent exploring the two objects, expressed in seconds, and the recognition index (time spent exploring the new object/total time spent exploring the new and familiar objects) are measured.
Similar exploration times with respect to the familiar object and to the new object reflect the fact that the familiar object has been forgotten, and therefore that there is a visual episodic memory deficit.

Conversely, a longer exploration time for the new object than for the familiar object reflects significant recollection of the familiar object and therefore an improvement in visual episodic memory.
A relatively low recognition index reflects the fact that the familiar object has been forgotten, and therefore that there is a visual episodic memory deficit.

Conversely, a high recognition index reflects significant recollection of the familiar object, and therefore an improvement in visual episodic memory.

The results which follow were obtained by forming the following four experimental groups, said groups receiving, respectively:
group 1: vehicle, methylcellulose+Tween alone)
group 2: 4-bromophenyl 1,4-diazabicyclo[3.2.2]nonane-4-carboxylate (2E)-but-2-enedioate, 0.1 mg/kg, p.o. (inactive dose)
group 3: rivastigmine, 0.03 mg/kg, p.o. (inactive dose)
group 4: 4-bromophenyl 1,4-diazabicyclo[3.2.2]nonane-4-carboxylate (2E)-but-2-enedioate, 0.01 mg/kg, p.o.+rivastigmine, 0.03 mg/kg, p.o. (inactive dose). The above doses are expressed in base equivalent.

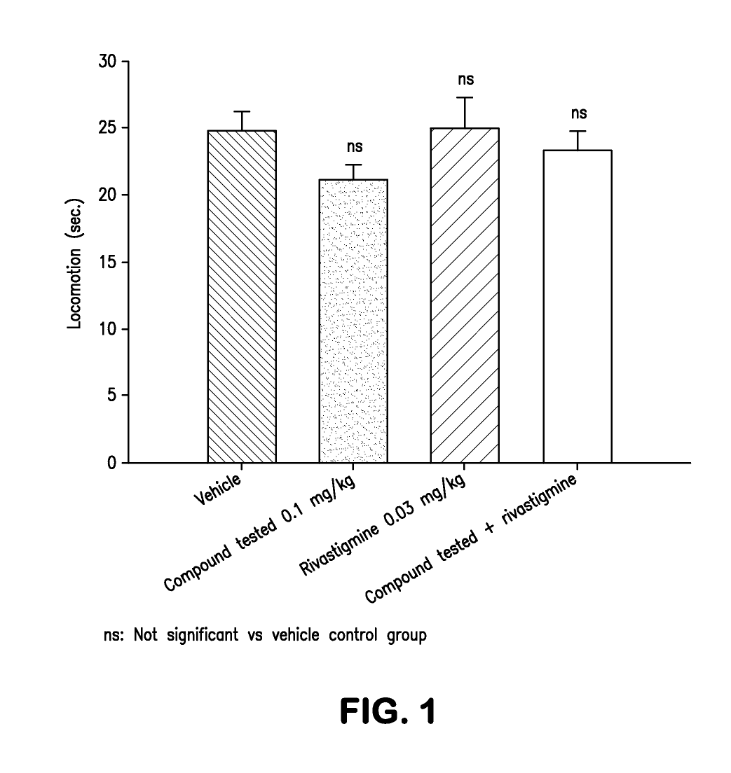

FIG. 1 and table 1 show the time spent in locomotion during the first accustomization session for each of the experimental groups.

TABLE 1

| Experimental group | Locomotion (seconds) |
|---|---|
| 1 | 24.80 ± 1.38 |
| 2 | 21.15 ± 1.14 |
| 3 | 24.94 ± 2.38 |
| 4 | 23.31 ± 1.40 |

After a single administration of each of the two compounds or of the combination of the two, no modification of the locomotion was observed during the session of accustomization to the environmental context.

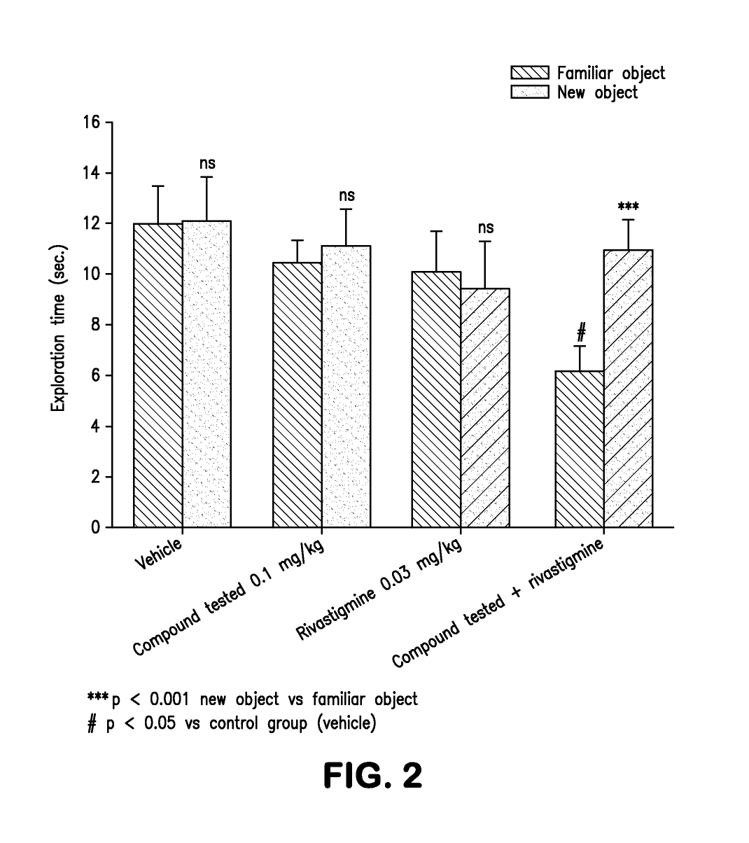

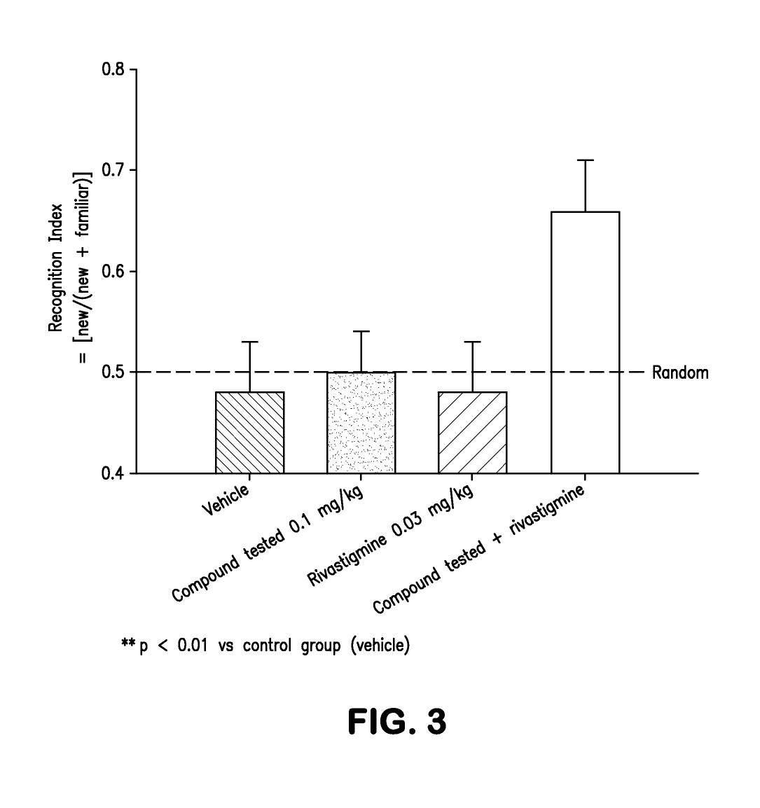

Table 2 in FIGS. 2 and 3 show:
the exploration time (in seconds) for the two objects, namely the new one and the familiar one; and
the recognition index as defined above;
   during the recalling session for each of the experimental groups.

During the recalling session, 24 hours after the learning session, the animals treated with the vehicle alone or with the inactive dose of each of the two compounds (1,4-diazabicyclo[3.2.2]nonane-4-carboxylate (2E)-but-2-enedioate or rivastigmine) show no significant discrimination between the familiar object and the new object. This reflects the fact that the familiar object has been forgotten.

Conversely, the animals treated with the combination of the inactive doses of each of the compounds tested show a very considerable exploration of the new object compared with the familiar object, reflecting significant recollection of the familiar object. This effect is observed on the exploration times for the two objects, expressed in seconds, and on the recognition index (table 2 and FIGS. 2 and 3).

TABLE 2

| | | | Recognition index |
|---|---|---|---|
| Experimental | Exploration time (seconds) | | (new/(new + |
| group | Familiar object | New object | familiar)) |
| 1 | 11.96 ± 1.51 | 12.09 ± 1.74 | 0.48 ± 0.05 |
| 2 | 10.47 ± 0.84 | 11.10 ± 1.44 | 0.50 ± 0.04 |
| 3 | 10.10 ± 1.56 | 9.42 ± 1.86 | 0.48 ± 0.05 |
| 4 | 6.14 ± 0.98 | 10.92 ± 1.18 | 0.66 ± 0.05 |

The results above show that, in a visual episodic memory test, the combination according to the invention clearly has a greater procognitive effect compared with the procognitive effect produced by the two active ingredients taken individually.

2. Test of short-term visual episodic memory deficit induced by intracerebroventricular (ICV) injections of β-amyloid protein 25-35 in mice

β-Amyloid peptides are the major constituents of the senile plaques observed in Alzheimer's disease. The Aβ25-35 peptide fragment contains the 11 amino acids necessary and sufficient for inducing neurological toxicity. It is for this reason that intracerebroventricular (ICV) injections of this peptide are considered to be a pathophysiological model of Alzheimer's disease with strong predictive validity.

The Aβ25-35 amyloid peptide is dissolved in distilled water at a final concentration of 3 mg/ml. It is then incubated at 37° C. for 4 days in order to form aggregates of toxic peptide. The control animals receive the scrambled peptide, consisting of the same amino acids, but organized according to a random sequence. This scrambled peptide exhibits no neuronal toxicity (nontoxic).

The peptide is injected intracerebroventricularly into CD1-strain mice anaesthetized with isoflurane. A microsyringe fitted with a 28-gauge needle 3 mm long is used. The needle is inserted unilaterally, 1 mm to the right of the central line equidistant from each eye and at an equal distance between the axis of the eyes and the axis of the ears. 3 μl of the aggregated peptide are injected over a period of one minute. The mice are used for the behavioral tests between 6 and 14 days after the intracerebral injection.

The behavioral experimental protocol used is comparable to that used in the spontaneous forgetting test in rats, described above.

1.) Accustomization phase: In this protocol, the mice are first subjected to a session of accustomization to the environmental context (light grey PVC chain of 52×52×40 cm, illumination at 6 Lux) for a period of 10 minutes. The time spent in active locomotion is measured using a timer.
2.) Learning phase: 24 hours later, two exactly identical objects (either metal triangles (5.5×3.3 cm), or a square red Lego brick (3.0×3.0×3.0 cm)) are placed in the experimental device. Each mouse is placed in the middle of the chamber, the nose against the wall opposite that where the objects/stimulus are located. The pairs of objects presented are alternated equitably in each of the groups of mice. The animals are left in the experimental chamber until they reach a time spent exploring the two objects equal to 10 seconds, out of a maximum period of 3 minutes. As soon as the 10 seconds of exploration are reached, the mouse is put back in its housing cage. This procedure makes it possible to homogenize the degree of impregnation of the animals for the familiar objects, and therefore to obtain a degree of learning which is comparable from one animal to the other.

3.) Recalling session: The recalling session takes place after a forgetting period of 1 hour for all the groups. This short forgetting period induces good recalling performance levels in the control animals and makes it possible to demonstrate a deficit induced by the injection of toxic peptide in the other animals. The mice are confronted with a pair of objects consisting of the object encountered during the learning phase and an unknown object. The mice are placed in the chamber in the same way as for the learning session.

This test lasts 5 minutes, during which the time spent exploring each of the 2 objects is measured.

For this short forgetting period, a control animal will exhibit greater exploration of the new object, reflecting significant recollection of the familiar object. Conversely, animals with a deficit will exhibit exploration times for the two objects which are not statistically different.

For the learning and recalling sessions, the exploration of an object is counted if the animal orients its nose toward the object, inside a perimeter of 2 cm around said object, or if it touches it with its nose or its feet. If the rat moves around the object or sits on it, these behaviors are not considered to be exploratory.

The raw data are expressed in seconds.

The compounds to be tested are administered intraperitoneally, 60 minutes before each of the three sessions of the protocol.

Five experimental groups were formed, receiving, respectively:

1—control scrambled peptide (nontoxic), vehicle
2—Aβ₂₅₋₃₅ peptide (toxic), vehicle
3—Aβ₂₅₋₃₅ peptide, acetylcholinesterase inhibitor (inactive dose)
4—Aβ₂₅₋₃₅ peptide, alpha-7 nicotinic receptor partial agonist (inactive dose)
5—Aβ₂₅₋₃₅ peptide, acetylcholinesterase inhibitor (inactive dose)+alpha-7 nicotinic receptor partial agonist (inactive dose).

During the recalling session, i.e. 1 hour after the learning session, the times spent exploring the two objects, expressed in seconds, are measured.
Similar exploration times with respect to the familiar object and to the new object reflect the fact that the familiar object has been forgotten, and therefore that there is complete abolition of the short-term recall performance levels.
Conversely, a longer exploration time for the new object compared with the familiar object reflects a good short-term memory recall performance level.

The recognition index (time spent exploring the new object/total time spent exploring the new and familiar objects) is also measured.
A relatively low recognition index reflects the fact that the familiar object has been completely forgotten, and therefore that there is complete abolition of the short-term recall performance levels.
Conversely, a high recognition index reflects a significant recollection of the familiar object and therefore a good short-term memory recall performance level.

The results which follow were obtained by forming the following five experimental groups, receiving, respectively:

1—control scrambled peptide (nontoxic), vehicle
2—Aβ₂₅₋₃₅ peptide (toxic), vehicle
3—Aβ₂₅₋₃₅ peptide, donepezil, 0.1 mg/kg (inactive dose)
4—Aβ₂₅₋₃₅ peptide, 4-bromophenyl 1,4-diazabicyclo[3.2.2]nonane-4-carboxylate (2E)-but-2-enedioate, 0.1 mg/kg (inactive dose)
5—Aβ₂₅₋₃₅ peptide, donepezil, 0.1 mg/kg+4-bromophenyl 1,4-diazabicyclo[3.2.2]nonane-4-carboxylate (2E)-but-2-enedioate, 0.1 mg/kg (inactive dose).

The above doses are expressed as base equivalent.

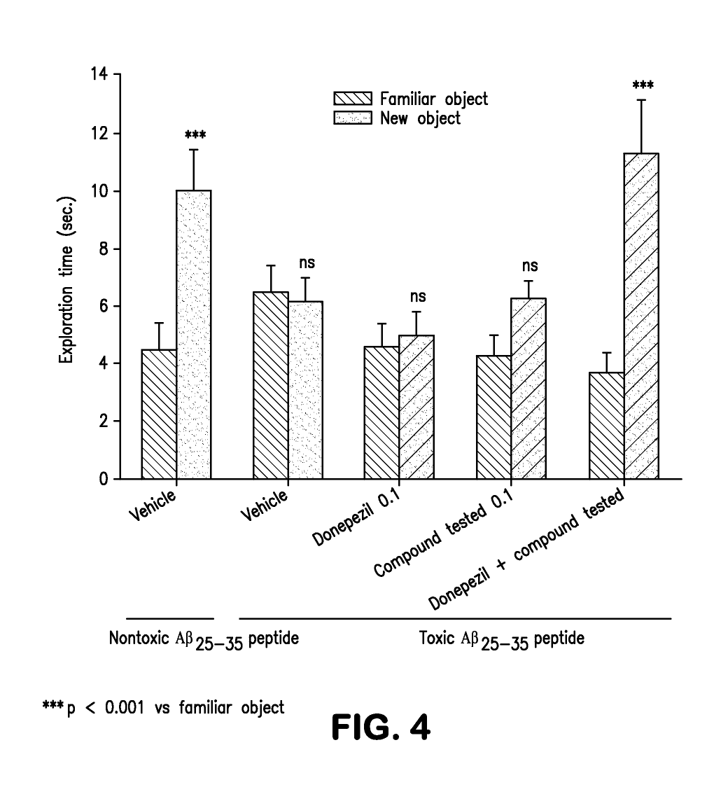

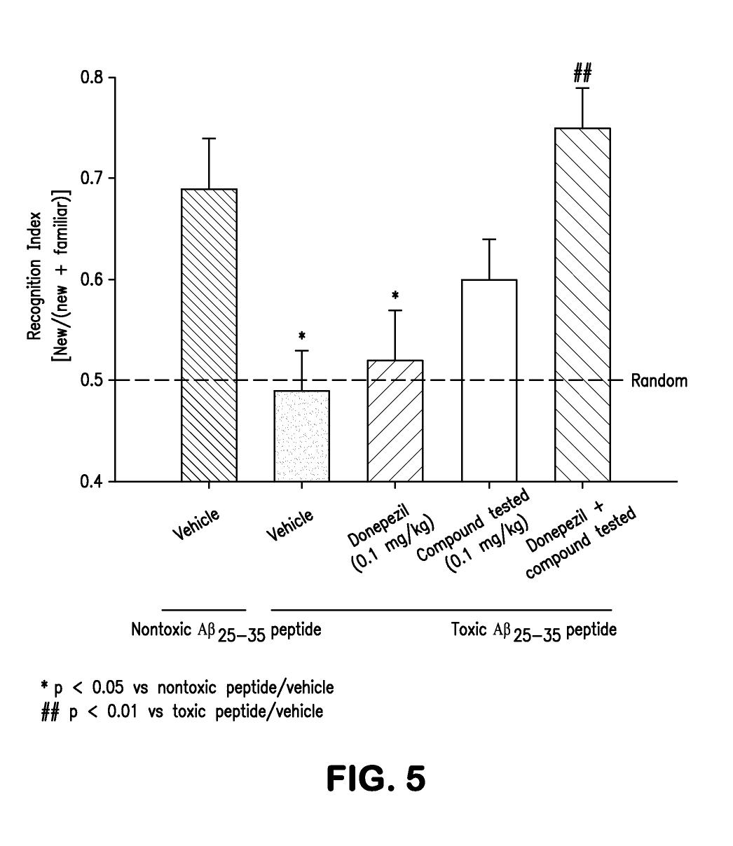

After a single administration of each of the two compounds or the combination of both, no modification of locomotion is observed during the session for accustomization to the environmental context. Similarly, no modification of the exploration times for the two identical objects during the learning session was reported. P Table 3 and FIGS. 4 and 5 show:
the exploration time (in seconds) for the two objects, namely the new object and the familiar object; and
the recognition index as defined above;
   during the recalling session for each of the experimental groups.

During the recalling session, the animals treated with the nontoxic control peptide exhibit intact short-term memory recall performance levels, resulting in significantly longer exploration of the new object compared with the familiar object. Conversely, the animals having received the toxic peptide show a complete abolition of the short-term recall performance levels, resulting in identical exploration of the familiar object and of the new object.

The dose of 4-bromophenyl 1,4-diazabicyclo[3.2.2]nonane-4-carboxylate (2E)-but-2-enedioate and the dose of donepezil are each inactive by themselves and do not result in an improvement in performance levels.

On the other hand, when these two products are coadministered, a very marked promnesic effect is observed, resulting in a return to short-term recall performance levels which are similar, or even superior, to those of the animals treated with the nontoxic peptide (table 3 and FIGS. 4 and 5).

TABLE 3

| | | | Recognition index |
|---|---|---|---|
| Experimental | Exploration time (seconds) | | (new/(new + |
| group | Familiar object | New object | familiar)) |
| 1 | 4.50 ± 0.90 | 10.00 ± 1.40 | 0.69 ± 0.05 |
| 2 | 6.50 ± 0.90 | 6.20 ± 0.80 | 0.49 ± 0.04 |
| 3 | 4.60 ± 0.70 | 5.00 ± 0.80 | 0.52 ± 0.05 |
| 4 | 4.30 ± 0.70 | 6.30 ± 0.60 | 0.60 ± 0.04 |
| 5 | 3.70 ± 0.70 | 11.30 ± 1.90 | 0.75 ± 0.04 |

The results above show that, in a test of short-term visual episodic memory deficit, the combination according to the invention results in a marked greater promnesic effect compared with the promnesic effect produced by the two active ingredients taken individually.

All the results obtained in the two series of experiments above demonstrate the greater effect of the combination according to the invention in terms of efficacy compared to each of the active ingredients administered alone and at the same dosage, on visual episodic memory disorders in an object recognition task in rodents. This greater effect is observed not only in animals with no deficit, the performance levels of which are improved, but also in a pathophysiological model of Alzheimer's disease, using intracerebral injections of toxic amyloid peptide.

This greater effect is representative of the efficacy of the combination according to the invention on cognitive disorders, and more particularly on cognitive disorders related to Alzheimer's disease, a pathological condition for which this type of cognitive function is known to be particularly impaired.

What is claimed is:

1. A combination of an alpha-7 nicotinic receptor partial agonist and an acetylcholinesterase inhibitor.

2. The combination according to claim 1, wherein the alpha-7 nicotinic receptor partial agonist is a compound of formula (I): 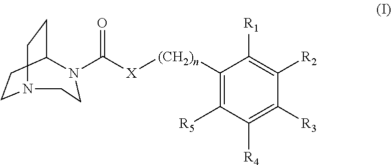
wherein:
X represents an oxygen atom or a group of formula NZ in which Z represents a hydrogen atom or a (C₁-C₆)alkyl group,
n represents the number 0, 1 or 2, and
R₁, R₂, R₃, R₄ and R₅ each represent, independently of one another, a hydrogen or halogen atom, or a trifluoromethyl group, a trifluoromethoxy group, a cyano group, a hydroxyl group, a (C₁-C₆)alkyl group, a (C₁-C₆)alkoxy group, a phenoxy group or a phenyl group which is optionally substituted with a halogen atom or a trifluoromethyl group, a cyano group, a hydroxyl group, a (C₁-C₆)alkyl group or a (C₁-C₆)alkoxy group, or else R₂ and R₃ together form a group of formula —OCH₂O— or —CH₂CH₂CH₂CH₂—,
or an acid addition salt, hydrate or solvate thereof.

3. The combination according to claim 2, wherein:
X represents an oxygen atom;
n represents the number 0;
R₁, R₂, R₄ and R₅ each represent a hydrogen atom; and
R₃ represents a halogen atom;
or an acid addition salt, hydrate or solvate thereof.

4. The combination according to claim 2, wherein the alpha-7 nicotinic receptor partial agonist is 4-bromophenyl 1,4-diazabicyclo[3.2.2]nonane-4-carboxylate, or an acid addition salt, hydrate or solvate thereof.

5. The combination according to claim 4, wherein the 4-bromophenyl 1,4-diazabicyclo[3.2.2]nonane-4-carboxylate is in the form of the (2E)-but-2-enedioate salt.

6. The combination according to claim 1, wherein the acetylcholinesterase inhibitor is chosen from rivastigmine and donepezil.

7. The combination according to claim 2, wherein the acetylcholinesterase inhibitor is chosen from rivastigmine and donepezil.

8. The combination according to claim 3, wherein the acetylcholinesterase inhibitor is chosen from rivastigmine and donepezil.

9. The combination according to claim 1, wherein the alpha-7 nicotinic receptor partial agonist is 4-bromophenyl 1,4-diazabicyclo[3.2.2]nonane-4-carboxylate (2E)-but-2-enedioate and the acetylcholinesterase inhibitor is rivastigmine.

10. The combination according to claim 1, wherein the alpha-7 nicotinic receptor partial agonist is 4-bromophenyl 1,4-diazabicyclo[3.2.2]nonane-4-carboxylate (2E)-but-2-enedioate and the acetylcholinesterase inhibitor is donepezil.

11. A pharmaceutical composition comprising the combination according to claim 1, and at least one pharmaceutically acceptable excipient.

12. A pharmaceutical composition comprising the combination according to claim 2, and at least one pharmaceutically acceptable excipient.

13. A pharmaceutical composition comprising the combination according to claim 3, and at least one pharmaceutically acceptable excipient.

14. A pharmaceutical composition comprising the combination according to claim 9, and at least one pharmaceutically acceptable excipient.

15. A pharmaceutical composition comprising the combination according to claim 10, and at least one pharmaceutically acceptable excipient.

16. A kit comprising an alpha-7 nicotinic receptor partial agonist and an acetylcholinesterase inhibitor, wherein the alpha-7 nicotinic receptor partial agonist and the acetylcholinesterase inhibitor are in separate compartments and being intended to be administered simultaneously, separately or spread out over time (sequential administration).

17. The kit according to claim 16, wherein the alpha-7 nicotinic receptor partial agonist is a compound of formula (I): 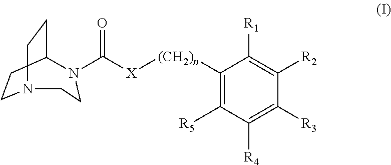
wherein:
X represents an oxygen atom or a group of formula NZ in which Z represents a hydrogen atom or a (C₁-C₆)alkyl group,
n represents the number 0, 1 or 2, and
R₁, R₂, R₃, R₄ and R₅ each represent, independently of one another, a hydrogen or halogen atom, or a trifluoromethyl group, a trifluoromethoxy group, a cyano group, a hydroxyl group, a (C₁-C₆)alkyl group, a (C₁-C₆)alkoxy group, a phenoxy group or a phenyl group which is optionally substituted with a halogen atom or a trifluoromethyl group, a cyano group, a hydroxyl group, a (C₁-C₆)alkyl group or a (C₁-C₆)alkoxy group, or else R₂ and R₃ together form a group of formula —OCH₂O— or —CH₂CH₂CH₂CH₂—,
or an acid addition salt, hydrate or solvate thereof.

18. The kit according to claim 16, wherein the alpha-7 nicotinic receptor partial agonist is 4-bromophenyl 1,4-diazabicyclo[3.2.2]nonane-4-carboxylate, or an acid addition salt, hydrate or solvate thereof.

19. A method of treating a cognitive disorder in a patient, comprising administering to said patient the combination according to claim 1.

20. A method of treating a cognitive disorder in a patient, comprising administering to said patient the combination according to claim 2.

21. A method of treating a cognitive disorder in a patient, comprising administering to said patient the combination according to claim 3.

22. A method of treating a cognitive disorder in a patient, comprising administering to said patient the combination according to claim 4.

23. A method of treating a cognitive disorder in a patient, comprising administering to said patient the combination according to claim 5.

24. A method of treating a cognitive disorder in a patient, comprising administering to said patient the combination according to claim 6.

25. A method of treating a cognitive disorder in a patient, comprising administering to said patient the combination according to claim 7.

26. A method of treating a cognitive disorder in a patient, comprising administering to said patient the combination according to claim 8.

27. A method of treating a cognitive disorder in a patient, comprising administering to said patient the combination according to claim 9.

28. The method according to claim 19, wherein the cognitive disorder is related to Alzheimer's disease.
